(19) United States
(12) Patent Application Publication
(10) Pub. No.: US 2012/0209064 A1
(43) Pub. Date: Aug. 16, 2012

(54) ENDOSCOPE APPARATUS AND METHOD OF SETTING REFERENCE IMAGE OF ENDOSCOPE APPARATUS

(75) Inventor: Kenji NUMATA, Tokyo (JP)

(73) Assignee: OLYMPUS CORPORATION, Tokyo (JP)

(21) Appl. No.: 13/368,744

(22) Filed: Feb. 8, 2012

(30) Foreign Application Priority Data
Feb. 14, 2011 (JP) 2011-028859
Feb. 14, 2011 (JP) 2011-028860

(51) Int. Cl. A61B 1/04

(52) U.S. Cl. 600/109

(57) ABSTRACT

An endoscope apparatus 1 includes a main body 4 to which a scope having an image pickup device at a distal end portion 8 of an insertion portion 7b is detachably attachable, a video signal processing section 22, a display section 5 and a control section 21. The control section 21 displays on the display section 5 a message for instructing a user to light-shield the distal end portion 8 according to an instruction for starting initialization processing to acquire a reference image of the image pickup device, acquires the reference image with the distal end portion 8 light-shielded and stores the acquired reference image in a memory 27.

### CROSS REFERENCE TO RELATED APPLICATION

This application claims benefit of Japanese Application No.2011-28859 filed in Japan on Feb. 14, 2011, and No. 2011-28860 filed in Japan on Feb. 14, 2011, the contents of which are incorporated by this reference.

### BACKGROUND OF THE INVENTION

1. Field of the Invention

The present invention relates to an endoscope apparatus and a method of setting a reference image of the endoscope apparatus.

2. Description of the Related Art

Endoscope apparatuses are being widely used in an industrial field and a medical field. There are endoscope apparatuses configured with a scope having an insertion portion with an image pickup device provided at a distal end portion thereof and a main body to which the scope is detachably connected.

A user can inspect an object by causing the distal end portion of the elongated insertion portion to approach a vicinity of the object and causing an image picked up by an image pickup device to be displayed on a monitor of the main body.

The image pickup device provided at the distal end portion may contain a sensitivity variation among pixels and pixel defects. For this reason, endoscope apparatuses are generally provided with a correction circuit to correct the sensitivity variation or the like.

Among prior arts, there is an endoscope apparatus provided with a correction circuit in a scope connected to a main body so that a sensitivity variation in the image pickup device of the scope or the like is corrected within the scope as proposed in Japanese Patent Application Laid-Open Publication No. 63-117727. According to the endoscope apparatus of the prior art, the inclusion of the correction circuit within the scope provides a merit of eliminating the need for an operation of correcting the sensitivity variation or the like in the main body when the scope is replaced.

Normally, the correction circuit includes a nonvolatile memory that stores a reference image for correction. The reference image is generated from a video signal of an image pickup device outputted by placing a scope in a space such as a dark room where light is shut out during manufacturing of the scope in a factory and driving the image pickup device in a predetermined thermostatic condition and the data thereof is recorded in a flash memory or the like of the correction circuit. Since the image pickup device has temperature dependency, the reference image should be acquired in the predetermined thermostatic condition.

When the endoscope apparatus is in use, a sensitivity variation between pixels and pixel defects or the like of the video signal captured by the scope are corrected using the reference image and supplied to the main body. As a result, the main body applies signal processing to a video signal whose pixel defects or the like are corrected and the object image is displayed on a monitor.

On the other hand, when the endoscope apparatus is used in a harsh working environment, the scope may be damaged or malfunction. In such a case, the damaged scope should be replaced.

In the case of the scope having the aforementioned correction circuit, since the scope newly connected through a replacement operation also has a correction circuit, a video signal whose pixel defects or the like are corrected is supplied to the main body and an image whose sensitivity variation or the like is corrected is displayed on the monitor.

### SUMMARY OF THE INVENTION

According to an aspect of the present invention, there can be provided an endoscope apparatus, including a main body to which a scope having an elongated insertion portion and an image pickup device at a distal end portion of the insertion portion is detachably attachable, a video signal processing section, provided in the main body, which drives the image pickup device and processes a video signal acquired by image pickup through the image pickup device, a display section, and a control section, provided in the main body, which displays on the display section a first message for instructing a user to perform light shielding of the distal end portion according to an instruction for starting initialization processing to acquire a reference image of the image pickup device, acquires the reference image with the distal end portion light-shielded and stores the acquired reference image in a predetermined memory.

According to another aspect of the present invention, there can be provided a method of setting a reference image of an endoscope apparatus, including displaying on a display section a first message for instructing a user to perform light-shielding of a distal end portion according to an instruction for starting initialization processing to acquire a reference image of an image pickup device provided at a distal end portion of a scope which is detachably attachable to a main body of the endoscope apparatus, acquiring the reference image with the distal end portion light-shielded and storing the acquired reference image in a predetermined memory.

According to yet another aspect of the present invention, there can be provided an endoscope apparatus including a main body to which a scope having an image pickup device at a distal end portion of an insertion portion is detachably attachable, a video signal processing section, provided in the main body, which drives the image pickup device and processes a video signal acquired by image pickup through the image pickup device, and a control section that displays on a display section a predetermined message to announce an instruction of light shielding of the distal end portion, heats the image pickup device after the predetermined message is displayed, drives the image pickup device with the image pickup device kept at a predetermined temperature, acquires a reference image and stores the reference image in a predetermined memory.

According to yet another aspect of the present invention, there can be provided a method of acquiring a reference image of an endoscope apparatus, including displaying on a display section a message for instructing light shielding of an image pickup section provided at a distal end portion of a scope so as to prevent light from entering the image pickup section, heating the image pickup section so that a temperature of the image pickup section reaches a predetermined temperature, driving the image pickup section with the temperature of the image pickup section kept at the predetermined temperature to acquire a reference image, and storing the acquired reference image in a predetermined memory.

### BRIEF DESCRIPTION OF THE DRAWINGS

FIG. 1 is an outside configuration diagram of an endoscope apparatus according to a first embodiment of the present invention;

FIG. 2 is a block diagram schematically showing a configuration of an endoscope apparatus 1 according to the first embodiment of the present invention;

FIG. 3 is a flowchart illustrating an example of a processing flow of initialization of the endoscope apparatus 1 according to the first embodiment of the present invention;

FIG. 4 is a flowchart illustrating an example of a processing flow of initialization of the endoscope apparatus 1 according to the first embodiment of the present invention;

FIG. 5 is a diagram illustrating a screen example for causing a user to instruct whether or not to perform initialization according to the first embodiment of the present invention;

FIG. 6 is a diagram illustrating a screen example of a scope connection instruction message when a scope 7 is not connected to a main body 4 according to the first embodiment of the present invention;

FIG. 7 is a diagram illustrating a screen example of a light-shielding instruction message according to the first embodiment of the present invention;

FIG. 8 is a diagram illustrating a screen example of a message for announcing that light-shielding is not performed correctly according to the first embodiment of the present invention;

FIG. 9 is a diagram illustrating a screen example instructing the user to wait for a predetermined time according to the first embodiment of the present invention;

FIG. 10 is a diagram illustrating a screen example for announcing normal termination of scope initialization to the user according to the first embodiment of the present invention;

FIG. 11 is a diagram illustrating a menu screen example including initialization processing according to a modification example of the first embodiment of the present invention;

FIG. 12 is a block diagram schematically illustrating a configuration of an endoscope apparatus 1A according to a second embodiment of the present invention;

FIG. 13 is a flowchart illustrating an example of a processing flow of initialization of the endoscope apparatus 1A according to the second embodiment of the present invention;

FIG. 14 is a flowchart illustrating an example of a processing flow of initialization of the endoscope apparatus 1A according to the second embodiment of the present invention;

FIG. 15 is a block diagram schematically illustrating a configuration of an endoscope apparatus 1B according to a third embodiment of the present invention;

FIG. 16 is a flowchart illustrating an example of part of a processing flow of an initialization operation of the endoscope apparatus 1B according to the third embodiment of the present invention; and

FIG. 17 is a flowchart illustrating an example of part of a processing flow of an initialization operation of the endoscope apparatus 1B according to a third embodiment of the present invention.

### DETAILED DESCRIPTION OF THE PREFERRED EMBODIMENTS

Hereinafter, embodiments of the present invention will be described with reference to the accompanying drawings.

### 1. Overall Configuration

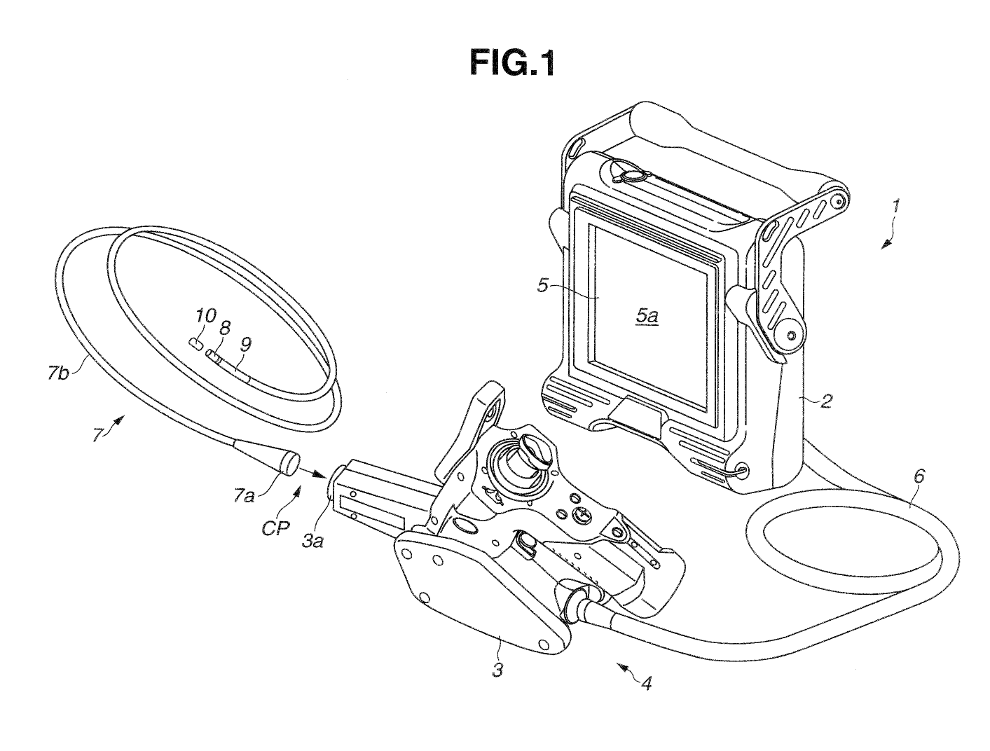

First, a configuration of an endoscope apparatus according to an embodiment of the present invention will be described based on FIG. 1. FIG. 1 is an outside configuration diagram of an endoscope apparatus according to an embodiment of the present invention.

As shown in FIG. 1, an endoscope apparatus 1 is configured by including a main unit 2 and an operation section 3 connected to the main unit 2. The main unit 2 and the operation section 3 constitute a main body 4 of the endoscope apparatus 1. The main unit 2 includes a display section 5 such as a liquid crystal display (LCD) as a display apparatus on which an endoscope image, operation menu or the like are displayed. The display section 5 as a monitor may also be a touch panel. The operation section 3 is connected to the main unit 2 via a universal cable 6 which is a connection cable.

A scope section (hereinafter also referred to as “scope”) 7 made up of a flexible insertion tube is detachably connected to the operation section 3. The scope 7 is detachably attachable to the operation section 3 at a connection point CP. A connector portion 7a of the scope 7 is connected to a connector section 3a of the operation section 3 and the scope 7 is thereby connected to the main body 4. The main body 4 is configured such that the scope 7 having an image pickup device at a distal end portion 8 of an insertion portion 7b is detachably attachable thereto.

An image pickup device (not shown), for example, a CMOS sensor, is incorporated in the distal end portion 8 of the insertion portion 7b of the scope 7 and an image pickup optical system such as a lens is arranged on the image pickup side of the image pickup device. A bending portion 9 is provided on the proximal end side of the distal end portion 8. A light-shielding adapter 10, which is a light-shielding member, can be attached to the distal end portion 8. The light-shielding adapter 10 is cap-shaped and is attachable to the distal end portion 8 so as to cover the distal end portion 8, and when the light-shielding adapter 10 is attached to the distal end portion 8, the image pickup device is light-shielded such that light does not enter the image pickup surface.

In the present embodiment, the light-shielding adapter 10 is cap-shaped so as to be attached to the distal end portion 8 as shown in FIG. 1, but may also be a device like a dark box into which the distal end portion 8 can be inserted or may also be an adhesive seal member made of a light-shielding material. In the case of the seal member, an adhesive material is provided on one side of the seal member and the image pickup device can be easily light-shielded by pasting the adhesive side thereof to an image pickup window for the image pickup section 12.

The operation section 3 is provided with various operation buttons such as a release button and an up/down/left/right (U/D/L/R) bending button. The user can pick up an image, record a still image of an object or the like by operating various types of operation buttons of the operation section 3. When the display section 5 is a touch panel, the user can perform various operations of the endoscope apparatus 1 (such as initialization) by operating the touch panel, for example, instructing operation contents of the endoscope apparatus 1, and therefore the display section itself constitutes an instruction section.

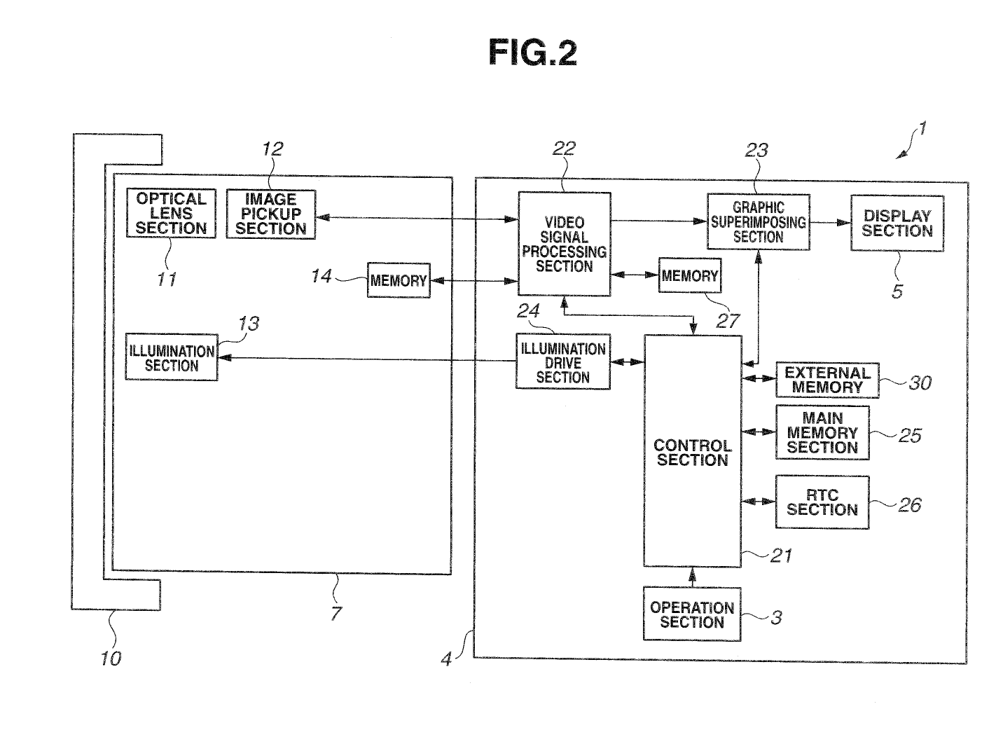

Image data obtained through image pickup is data to be inspected, recorded in a recording medium such as a memory card, and an external memory 30 (FIG. 2) which is the recording medium is detachably attachable to the main unit 2.

The operation section 3 is detachably attachable to the main unit 2.

As will be described later, the scope 7 has an identification section for identification of its type when connected to the main body 4. The main body 4 is configured, when the scope 7 is connected thereto, so as to detect or read the state or identification data (that is, ID data) and identify its type. Here, the ID includes not only information on the type such as model of the apparatus but also unique information such as serial number for individual identification.

As described above, the scope 7 is detachably attachable to the operation section 3 (main body 4), and when the scope 7 is damaged, only the scope 7 can be replaced.

Furthermore, in the case of FIG. 1, the main unit 2 and the operation section 3 are connected via the universal cable 6, but the main unit 2 and the operation section 3 may be united into one unit. In such a case, the scope 7 is detachably attachable to the apparatus (main body), which is the one unit.

### 2. Circuit Configuration

FIG. 2 is a block diagram schematically illustrating a configuration of the endoscope apparatus 1. As described above, the endoscope apparatus 1 is configured by the main body 4 and the scope 7 and the light-shielding adapter 10 can be attached to the distal end of the scope 7.

The scope 7 is configured by including an optical lens section 11, an image pickup section 12, an illumination section 13 and a memory 14.

The optical lens section 11 is an objective optical system provided at the distal end portion 8 to receive reflected light from an object.

The image pickup section 12 is an image pickup device having an image pickup surface such as a CMOS sensor and is arranged in the distal end portion 8 such that the image pickup surface is located at the focus of the optical lens section 11. The image pickup section 12 drives the image pickup device based on a drive signal from the main body 4 and outputs a photoelectrically converted video signal to the main body 4.

The illumination section 13 is provided at the distal end portion and configured by including a light-emitting element that emits illumination light to the object. The light-emitting element is, for example, an LED (light-emitting diode). The illumination section 13 is driven based on a drive signal from the main body 4 and emits illumination light to the object.

The memory 14 is a nonvolatile memory that stores information on an identifier (hereinafter referred to as “ID”) for type and individual identification of the scope 7.

The main body 4 is configured by including the operation section 3, the display section 5, a control section 21, a video signal processing section 22, a graphic superimposing section 23, an illumination drive section 24, a main memory section 25, a real-time clock (RTC) section 26 and a memory 27.

The control section 21 includes a central processing unit (CPU) and is a processing section that controls overall operations of the various circuits in the main body 4. The control section 21 performs processing corresponding to the user's operation on the operation section 3 (or touch panel).

The video signal processing section 22 operates based on a control signal from the control section 21, supplies a drive signal to the image pickup section 12, receives a video signal from the image pickup section 12, applies predetermined image processing to the received video signal and outputs the signal to the display section 5 via the graphic superimposing section 23. Furthermore, the video signal processing section 22 stores a video signal in the memory 27 based on a control signal from the control section 21 or reads a video signal from the memory 27 and outputs the video signal to the display section 5. That is, the video signal processing section 22 is a circuit provided in the main body 4 that drives the image pickup device and processes the video signal acquired by image pickup through the image pickup device.

The graphic superimposing section 23 displays a menu screen or the like on the screen 5a of the display section 5 based on a control signal from the control section 21 and a video signal from the video signal processing section 22 or outputs a video signal resulting from superimposing predetermined information on an object image to the screen 5a of the display section 5.

The illumination drive section 24 outputs a drive signal to the illumination section 13 based on a control signal from the control section 21.

The main memory section 25 includes a ROM that stores a program for various functions, a RAM used as a work area when the CPU in the control section 21 executes the program and a nonvolatile memory such as a flash memory. Therefore, the CPU of the control section 21 reads the program in the main memory section 25 according to an operation signal from the operation section 3 and executes the program while using the RAM. The nonvolatile memory records information such as an identifier (hereinafter referred to as “ID”) for individual identification of the connected scope 7. As will be described later, when power is turned on, the control section 21 records the information on the ID of the scope, which is or was connected, in the nonvolatile memory to determine whether or not the scope 7 is replaced.

The real-time clock section 26 is a circuit that generates and stores a time recorded when an endoscope image is recorded.

The external memory 30 is a nonvolatile memory for recording a video signal acquired by image pickup by the image pickup section 12. The control section 21 can also cause an image recorded in the external memory 30 to be displayed on the screen 5a of the display section 5 via the video signal processing section 22.

### 3. Operation

Next, an initialization operation in the endoscope apparatus 1, and an operation of acquiring a reference image in particular will be described.

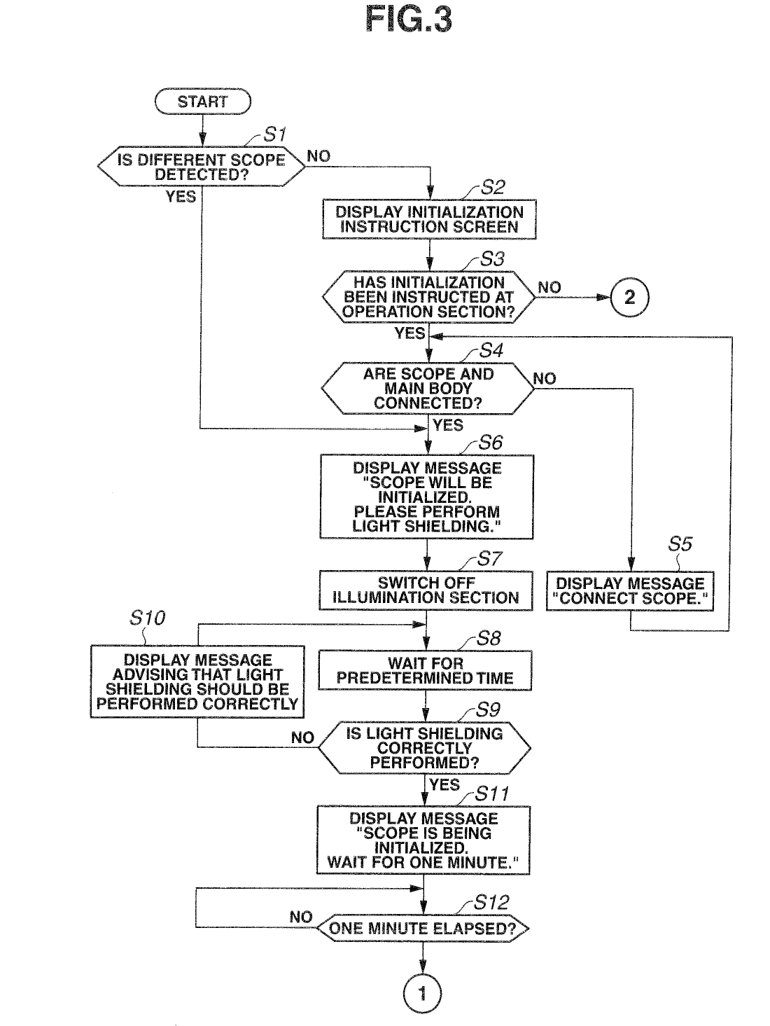

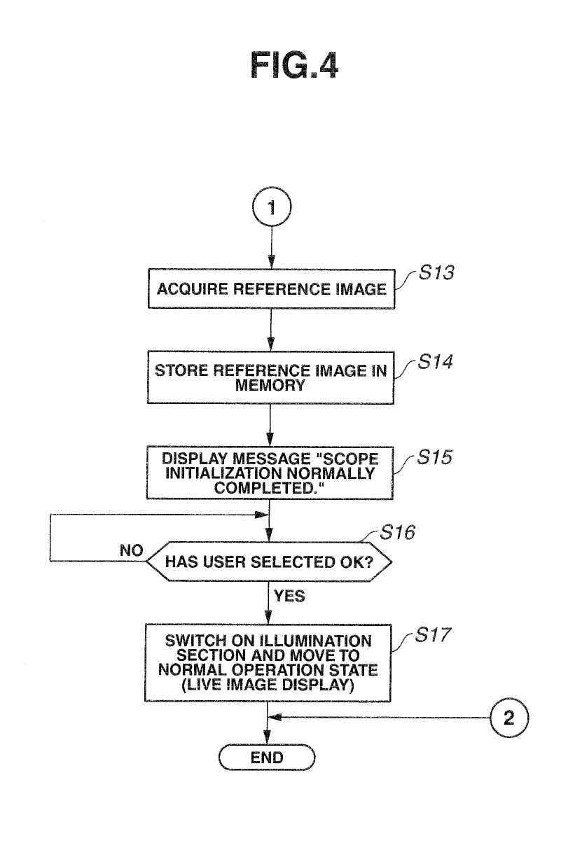

When the scope 7 is replaced, the user first turns off the power to the endoscope apparatus 1 and replaces the scope 7 in the power-off state. After the replacement, the user turns on the power to the endoscope apparatus 1, and then processing in FIG. 3 and FIG. 4 is executed.

FIG. 3 and FIG. 4 show flowcharts illustrating an example of a processing flow of initialization of the endoscope apparatus 1. The processing in FIG. 3 and FIG. 4 is executed by the CPU of the control section 21.

First, when the power to the endoscope apparatus 1 is turned on, the control section 21 determines whether or not a scope different from the previously connected scope is detected (S1). This determination is made by comparing the scope ID recorded in the memory 14 provided in the scope 7 with the scope ID recorded in the main memory section 25.

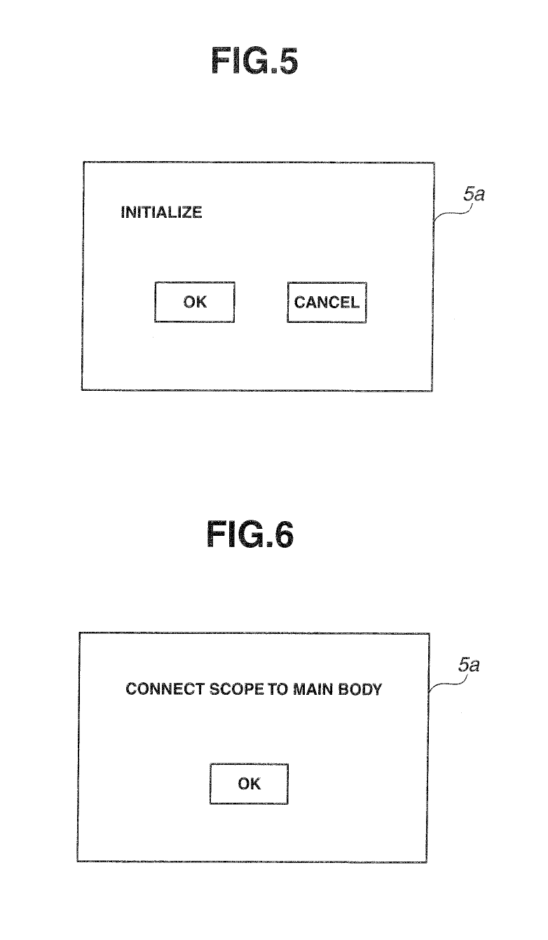

When no different scope is detected (S1: NO), the control section 21 displays a screen for checking the execution of initialization on the screen 5a of the display section 5 and determines, based on the display, whether or not the user has instructed initialization through the operation section 3. That is, upon detecting that the scope connected when the power of the main body 4 of the endoscope apparatus 1 is turned on is different from the scope connected when the main body 4 was turned off last time, the control section 21 displays an initialization instruction screen (FIG. 5) on the screen 5a of the display section 5.

FIG. 5 is a diagram illustrating a screen example to cause the user to instruct whether or not to perform initialization. When no different scope is connected to the main body 4 (S1: NO), the control section 21 reads the screen data recorded in the ROM of the main memory section 25 and displays the screen in FIG. 5 on the screen 5a of the display section 5 (S2). When the user selects an “OK” button on the screen, initialization is instructed (S3: YES) and when the user selects a “cancel” button, initialization is not instructed (S3: NO). When initialization is not instructed (S3: NO), the process ends. That is, the control section 21 displays the initialization instruction screen (FIG. 5) on the screen 5a of the display section 5 for causing the user to instruct starting the initialization processing to acquire a reference image of the image pickup device provided at the distal end portion 8 of the scope 7.

When initialization is instructed (S3: YES), the control section 21 determines whether or not the scope 7 and the main body 4 are connected (S4). Whether or not the scope 7 and the main body 4 are connected can be determined by checking whether or not the scope ID can be read, or the like.

When the scope 7 is not connected to the main body 4 (S4: NO), the control section 21 displays a scope connection instruction message “Please connect the scope to the main body” on the screen 5a of the display section 5 (S5), and the process returns to S3. That is, upon detecting that the scope 7 is not connected to the main body 4 before acquiring a reference image, the control section 21 displays a message for instructing a connection of the scope 7 to the main body 4 on the screen 5a of the display section 5.

FIG. 6 is a diagram illustrating a screen example of a scope connection instruction message when the scope 7 is not connected to the main body 4. As shown in FIG. 6, the message and the OK″ button are displayed on the screen 5a.

When a different scope is detected (S1: YES) or when the scope 7 is connected to the main body 4 (S4: YES), the control section 21 displays a light-shielding instruction message “Scope initialization will be performed. Please perform light shielding of the distal end portion of the scope” on the screen 5a of the display section 5 (S6). That is, the control section 21 displays on the screen 5a of the display section 5 a message for instructing attachment of the light-shielding member to light-shield the image pickup section 12 so as to prevent light from entering the image pickup section 12 provided at the distal end portion 8 of the scope 7.

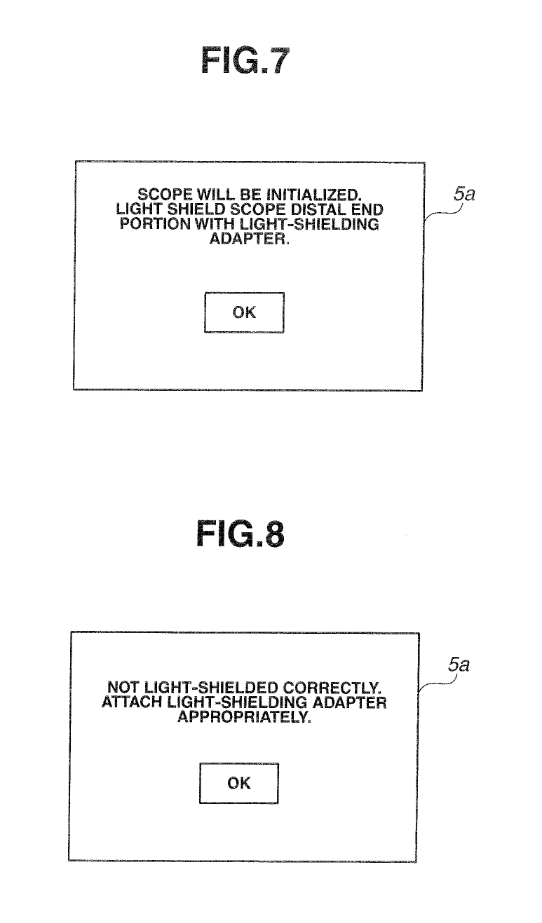

FIG. 7 is a diagram illustrating a screen example of a light-shielding instruction message. As shown in FIG. 7, the message and the “OK” button are displayed on the screen 5a. That is, the control section 21 displays on the screen 5a of the display section 5 a message to instruct the user to perform light shielding of the distal end portion 8 according to an instruction for starting initialization processing.

Since the display of FIG. 7 is shown on the screen 5a of the display section 5, the user attaches the light-shielding adapter 10 to the distal end portion 8 of the scope 7. After the attachment, the user can select the OK button. In consideration of the operation time required to attach the light-shielding adapter 10 to the distal end portion 8 of the scope 7, the control section 21 waits for a predetermined time (S8). After a lapse of the predetermined time, the control section 21 determines whether or not light shielding of the scope 7 is correctly performed (S9).

Whether or not light shielding is correctly performed can be determined by driving the image pickup section 12 and determining whether or not there are a predetermined number of or more pixel outputs equal to or above a predetermined threshold TH. This is because when there are pixel outputs equal to or above the predetermined threshold TH, it is assumed that the image pickup device is receiving light.

When light shielding is not correctly performed (S9: NO), the control section 21 displays a message advising that light shielding should be performed correctly on the screen 5a of the display section 5 (S10), and the process moves to S8. That is, after displaying the message in FIG. 7, the control section 21 determines the light-shielding state of the distal end portion 8 and displays, when determining that the distal end portion 8 is not light-shielded correctly, a message instructing the user to perform light shielding of the distal end portion 8 correctly on the screen 5a of the display section 5.

As for the message for instructing light shielding of the distal end portion 8, instead of the message in FIG. 7 “The scope will be initialized. Light shield the scope distal end portion with the light-shielding adapter,” the message may be like: “Not light-shielded correctly,” “(In the first place) Is light shielding really performed?,” “Can a reference image be acquired (in this condition)?,” or “Is a reference image ready to be acquired?,” and so forth.

FIG. 8 is a diagram illustrating a screen example of a message announcing that light shielding is not performed correctly. As shown in FIG. 8, the message and the “OK” button are displayed. As shown in FIG. 8, since a message “Not light-shielded correctly. Attach the light-shielding adapter appropriately” is displayed on the screen 5a of the display section 5, the user can attach the light-shielding adapter 10 to the distal end portion 8 of the scope 7 appropriately. After the attachment, the user selects the “OK” button. With this, the control section 21 is instructed to complete the light-shielding operation.

Such a connection check (S9) and screen display (S10) are performed because acquiring a reference image is not an operation frequently performed by the user, and therefore it is intended to check to see whether or not the operation is reliably performed so that the reference image may be acquired reliably in the subsequent processing.

When it is determined that light shielding is correctly performed (S9: YES), the control section 21 displays a message “The scope is being initialized; please wait for one minute” on the screen 5a of the display section (S11). That is, a message to instruct the user to wait for a predetermined time is displayed (instructed). In other words, the control section 21 displays on the screen 5a of the display section 5 a message announcing the time required to acquire a reference image to the user.

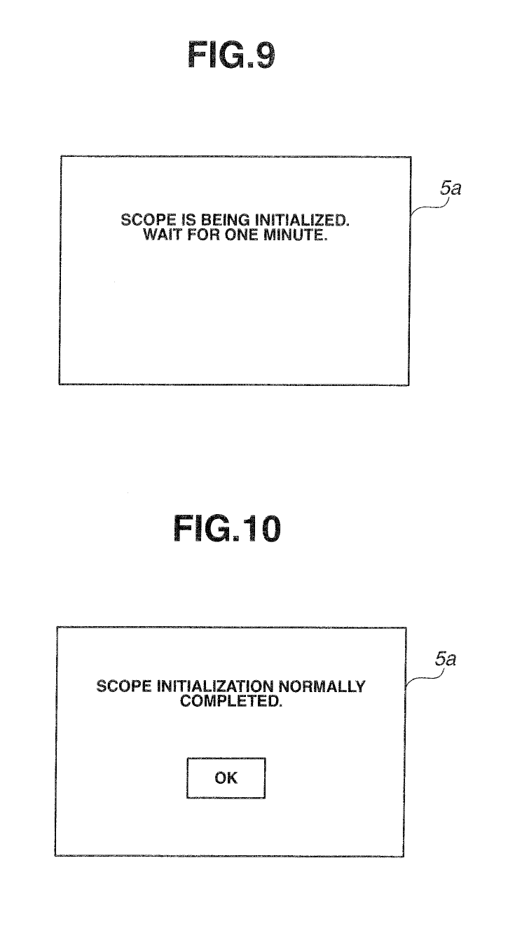

FIG. 9 is a diagram illustrating a screen example displaying a message for instructing the user to wait for a predetermined time.

Regarding the predetermined time, a time period after the scope 7 is newly connected to the main body 4 until the temperature of the image pickup device of the scope 7 reaches a temperature in a steady state is measured through an experiment or the like and known beforehand. Therefore, the time is set based on a time period required for the hard member of the distal end portion 8 of the scope 7 to reach a state equivalent to a steady state through thermal conduction under a normal room temperature.

When a reference image is acquired after setting the image pickup section 12 to a temperature in a steady state, for example, 50±1° C., the image pickup section 12 is set to a predetermined temperature by placing a heating section such as a heater in the distal end portion 8 and passing a predetermined current into the heating section. In such a case, the time in the message in FIG. 9 is determined and displayed by also taking into account the time after starting heating until the temperature of the heating section reaches the predetermined temperature. That is, the time required to acquire a reference image included in the message in FIG. 9 includes a time period after the image pickup device is heated by the heater provided at the distal end portion 8 of the scope 7 until the temperature reaches the predetermined temperature.

As the method of heating the image pickup section 12 to the predetermined temperature, instead of using the heating section such as a heater, for example, the illumination section 13 may be driven to increase the temperature of the image pickup section 12 to the predetermined temperature or the image pickup section 12 may be driven in a high power consumption mode so that the amount of power consumption of the image pickup section 12 increases so that the temperature of the image pickup section 12 itself rises to the predetermined temperature.

When a predetermined time elapses after the message in FIG. 9 is displayed, the control section 21 drives the image pickup section 12 with the distal end portion light-shielded and acquires a reference image (S13). The control section 21 then displays on the screen 5a of the display section 5 a message announcing to the user that initialization processing is in progress before acquiring the reference image. That is, the screen in FIG. 9 is displayed at least until the reference image is acquired after the “OK” button is selected on the screen in FIG. 7 or FIG. 8 before acquiring the reference image.

Here, the acquisition of the reference image includes the acquisition of a fixed pattern noise image to correct a sensitivity variation between pixels and the acquisition of position information of defective pixels on the image pickup surface and the luminance level. Therefore, the reference image includes the fixed pattern noise image, the position information of defective pixels and the luminance level. The acquisition of the reference image may be acquisition of any one of the fixed pattern noise image, position information of defective pixels and the luminance level depending on the required image quality or the like.

The control section 21 stores and records the reference image acquired by image pickup by the image pickup section 12 in the memory 27 (S14).

The reference image may be acquired a plurality of times by the image pickup device, and data of an average value or maximum value of the plurality of images acquired may be used as the reference image.

Moreover, the control section 21 may store the reference image not in the memory 27 but in the memory 14.

After the reference image is stored, the control section 21 displays on the screen 5a of the display section 5 a message “Scope initialization is completed normally” (S15). That is, after the reference image is stored in the predetermined memory, the control section 21 displays on the screen 5a of the display section 5 an initialization completion screen (FIG. 10) to announce to the user that initialization processing is completed.

FIG. 10 is a diagram illustrating a screen example for announcing to the user that the scope initialization is completed normally. As shown in FIG. 10, a message “Scope initialization is normally completed” and the “OK” button are displayed on the screen 5a.

The control section 21 determines whether or not the user selects the “OK” button on the screen (S16). When the user selects the “OK” button on the screen (S16: YES), the control section 21 drives the illumination section 13 to turn it on, and the process moves to a normal operation state, that is, a state in which a live image is displayed on the screen 5a of the display section 5 (S17), and then the user can use the endoscope apparatus 1 for an inspection operation. If the user does not select the “OK” button on the screen (S16: NO), the control section 21 performs no processing.

The message as shown in FIG. 10 is displayed because if the initialization is completed without any display (consultation required) and a normal operation state is displayed on the screen 5a of the display section 5, the user cannot know whether or not initialization is correctly performed. Furthermore, the user is instructed to select the “OK” button also for the purpose of making the user confirm that initialization is completed.

The reference image recorded in the memory 27 of the main body 4 is used by the video signal processing section 22 of the main body 4 to correct a sensitivity variation between pixels and pixel defects or the like, and clear video signals whose sensitivity variation between pixels or the like is corrected are outputted to the screen 5a of the display section 5.

Conventionally, the reference image is acquired in a thermostatic chamber in a factory or the like during manufacturing of the scope, and cannot be acquired reliably by the user.

According to the endoscope apparatus of the present embodiment, the user can reliably perform a reference image acquiring operation and acquire an accurate reference image.

### 4. Modification Examples

Next, modification examples of the aforementioned embodiment will be described.

### Modification Example 1

According to the aforementioned embodiment, the initialization processing is performed in the case where a different scope is detected when the power to the endoscope apparatus 1 is turned on first and a different scope is then detected, and in the case where no different scope is detected and initialization is instructed.

Without being limited thereto, a menu screen may be displayed separately when the power to the endoscope apparatus 1 is turned on and the endoscope apparatus 1 is operating in a normal operation state, that is, while a live image is being displayed on the screen 5a of the display section 5, the user may be instructed to select initialization from the menu screen so that the initialization processing from S5 onward is performed.

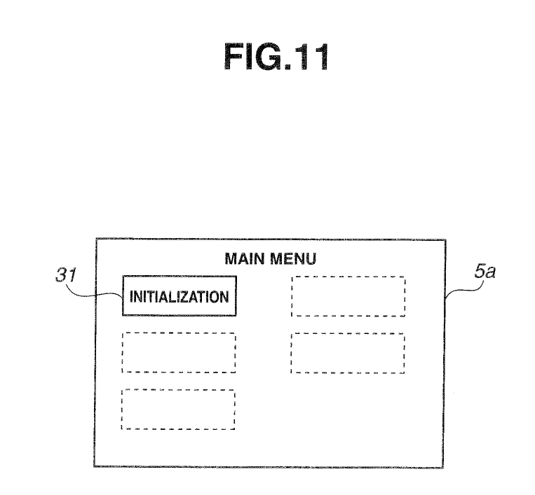

FIG. 11 is a diagram illustrating a menu screen example including the initialization processing. As shown in FIG. 10, an “initialization” button 31 for acquiring the aforementioned reference image is included in a predetermined menu screen, for example, a main menu screen, and the initialization processing from S4 onward in FIG. 3 may be executed when the user selects the button 31. That is, the menu screen in FIG. 11 constitutes an initialization instruction screen to cause the user to instruct starting the initialization processing including the acquisition of a reference image of the image pickup device.

### Modification Example 2

In the aforementioned embodiment, a new scope is mounted after turning off the power to the endoscope apparatus 1.

Without being limited thereto, however, in the case of an endoscope apparatus that allows the scope 7 to be removed or attached without turning off the power to the endoscope apparatus 1, that is, in a hot line condition, the attachment of the scope after removal may be detected and the aforementioned initialization processing may be performed. That is, when the scope 7 is replaced while the power to the main body 4 of the endoscope apparatus 1 is on, the control section 21 may display the initialization instruction screen (FIG. 5) on the screen of the display section.

For example, when the user replaces the scope without turning off the power to the endoscope apparatus 1 during an endoscope inspection, the control section 21 may execute the initialization processing from S6 onward upon detecting the attachment of the scope after removal.

Conventionally, the reference image is acquired in a thermostatic chamber in a factory or the like during manufacturing of the scope, and cannot be acquired accurately and reliably by the user.

According to the aforementioned embodiment and modification examples, the user can acquire a reference image accurately and reliably in any place other than a factory or the like.

### SECOND EMBODIMENT

Next, a configuration of an endoscope apparatus according to a second embodiment of the present invention will be described.

### 1. Configuration

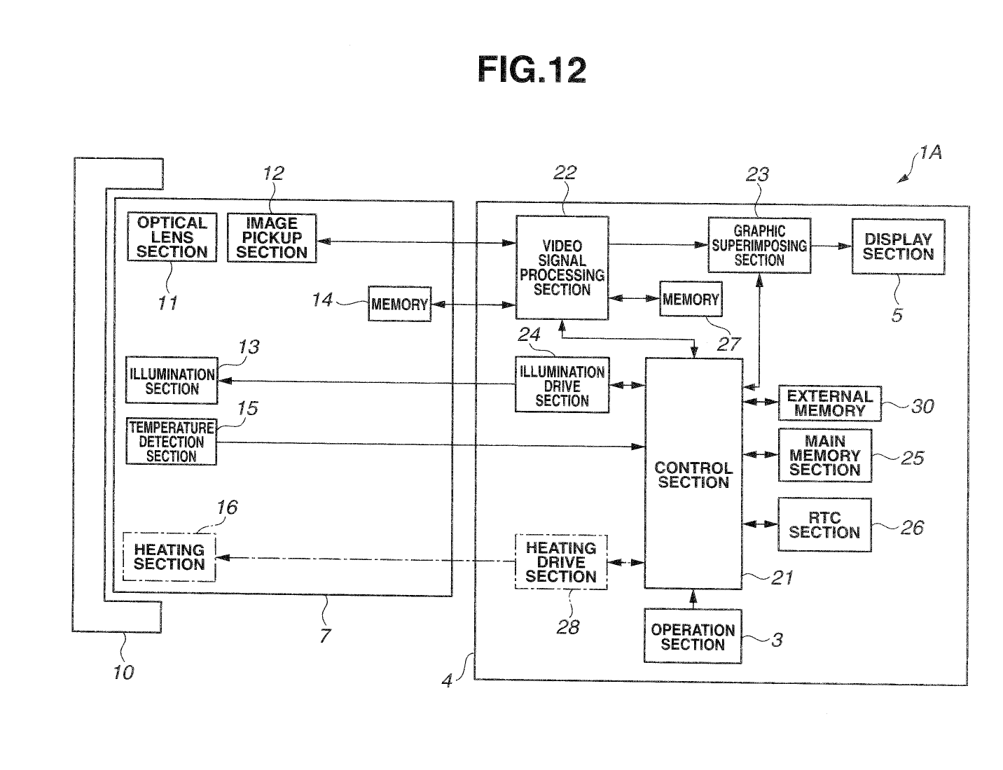

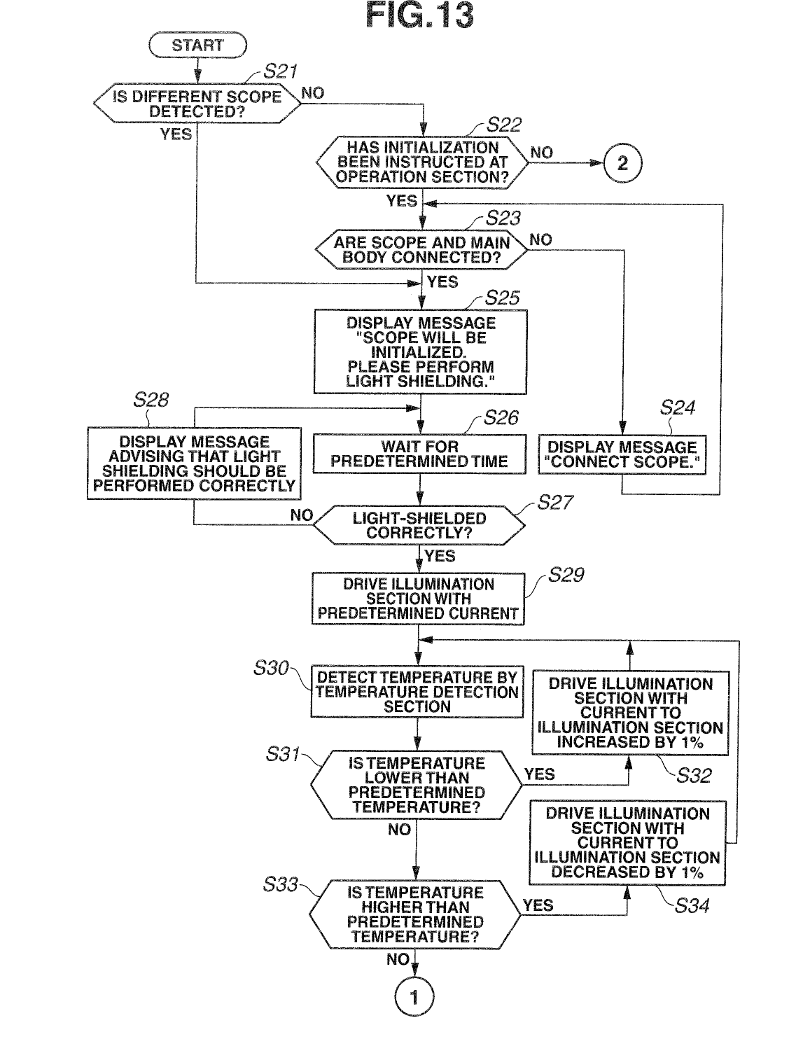

An outside view of the endoscope apparatus according to the second embodiment is similar to that in FIG. 1 of the first embodiment. The configuration of the endoscope apparatus according to the second embodiment is also similar to the configuration in FIG. 2, but has a different configuration as shown in FIG. 12. Therefore, in the second embodiment, the same components as those in the first embodiment will be assigned the same reference numerals and descriptions thereof will be omitted.

FIG. 12 is a block diagram schematically illustrating a configuration of an endoscope apparatus 1A of the present embodiment. As described above, the endoscope apparatus 1A is constructed of the main body 4 and the scope 7, and the light-shielding adapter 10 can be attached to the distal end of the scope 7.

The scope 7 is configured by including the optical lens section 11, the image pickup section 12, the illumination section 13, the memory 14 and a temperature detection section 15.

The temperature detection section 15 is provided especially in the vicinity of the image pickup device of the image pickup section 12 in the distal end portion 8 and is configured by including a temperature detection element for detecting a temperature of the image pickup section 12. The temperature detection section 15 supplies the output of the temperature detection element to the main body 4.

### 2. Operation

Next, an initialization operation by the endoscope apparatus 1A, especially a reference image acquiring operation will be described.

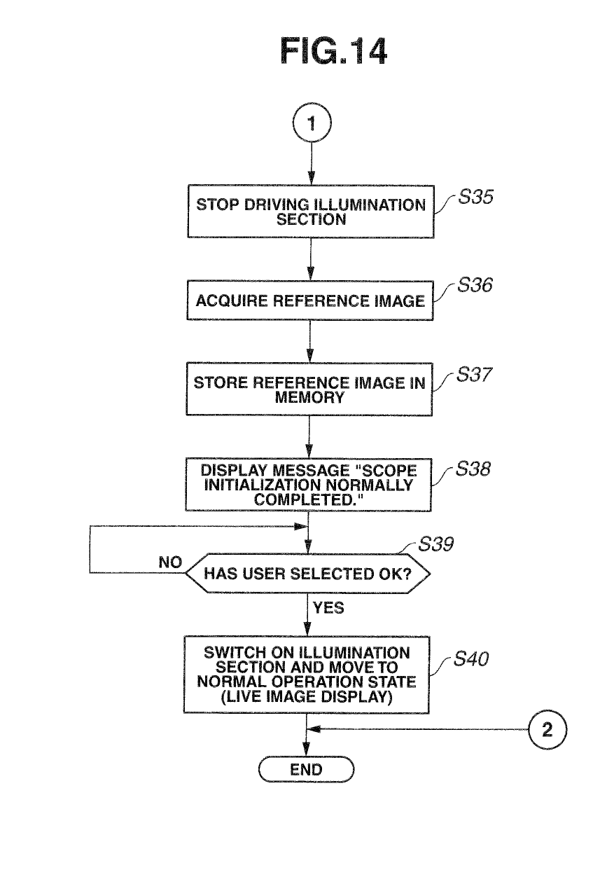

In replacing the scope 7, the user turns off the power to the endoscope apparatus 1A first and replaces the scope 7 in the off state. When the user turns on the power to the endoscope apparatus 1A after the replacement, the processing in FIG. 13 and FIG. 14 is executed.

FIG. 13 and FIG. 14 are flowcharts illustrating a flow example of the initialization processing by the endoscope apparatus 1A. The processing in FIG. 13 and FIG. 14 is performed by the CPU of the control section 21.

First, when the power to the endoscope apparatus 1A is turned on, the control section 21 determines whether or not a scope different from the previously connected scope is detected (S21). This determination can be made by comparing the ID of the scope recorded in the memory 14 provided in the scope 7 with the ID of the scope recorded in the main memory section 25.

When no different scope is detected (S21: NO), the control section 21 determines whether or not initialization is instructed at the operation section 3 (S22). The control section 21 reads screen data recorded in the ROM of the main memory section 25 and displays the message shown in FIG. 5 on the screen 5a of the display section 5. When the user selects the “cancel” button on the screen, the initialization is not instructed (S22: NO). When the initialization is not instructed (S22: NO), the process ends.

When the user selects the “OK” button on the screen, the initialization is instructed (S22: YES), whereas when the user selects the “cancel” button, the initialization is not instructed (S22: NO).

When the initialization is instructed (S22: YES), the control section 21 determines whether or not the scope 7 and the main body 4 are connected (S23). Whether or not the scope 7 and the main body 4 are connected can be determined by checking whether or not the ID of the scope can be read or the like.

When the scope 7 is not connected to the main body 4 (S23: NO), the control section 21 displays the message in FIG. 6 “Connect the scope to the main body” on the screen 5a of the display section 5 (S24), and the process returns to S23.

When a different scope is detected (S21: YES) or when the scope 7 is connected to the main body 4 (S23: YES), the control section 21 displays the message in FIG. 7 “Scope initialization will be performed. Please light-shield the distal end portion of the scope” on the screen 5a of the display section 5 (S25). That is, the control section 21 displays on the screen 5a of the display section 5 a message for instructing attachment of a light-shielding member for light-shielding the image pickup section 12 so as to prevent light from entering the image pickup section 12 provided at the distal end portion 8 of the scope 7.

Since such a display appears on the screen 5a of the display section 5, the user attaches the light-shielding adapter 10 to the distal end portion 8 of the scope 7. The control section 21 waits for a predetermined time in consideration of the operation time for attaching the light-shielding adapter 10 to the distal end portion 8 of the scope 7 (S26).

When a predetermined time elapses, the control section 21 determines whether or not the scope 7 is correctly light-shielded (S27).

Whether or not light-shielding is correctly performed can be determined by driving the image pickup section 12 and determining whether or not there are a predetermined number of or more pixel outputs equal to or above a predetermined threshold TH. This is because when there are pixel outputs equal to or above the predetermined threshold TH, it is assumed that the image pickup device is receiving light.

When light-shielding is not correctly performed (S27: NO), the control section 21 displays on the screen 5a of the display section 5 a message instructing that light-shielding should be performed correctly (S28), and the process moves to S26.

When it is determined that light-shielding is performed correctly (S27: YES), the control section 21 drives the illumination section 13 with a predetermined current (S29). The illumination section 13 generates heat when it is driven. When driving of the illumination section 13 with a predetermined current is started, the image pickup section 12 (to be more specific, the image pickup device) provided in the vicinity of the illumination section 13 is heated and the temperature of the image pickup device starts to rise.

The control section 21 detects the temperature through the temperature detection section 15 (S30). The temperature detected by the temperature detection section 15 substantially corresponds to the temperature of the image pickup section 12.

The control section 21 determines whether or not the detected temperature is lower than a predetermined temperature, for example, 50±1° C. (S31).

When the detected temperature is lower than the predetermined temperature (S31: YES), the control section 21 increases the current of the drive signal to the illumination section 13 by a predetermined amount, for example, 1% here, and drives the illumination section 13 (S32), and the process moves to S30.

When the detected temperature is not lower than the predetermined temperature (S31: NO), the control section 21 decreases the current of the drive signal to the illumination section 13 by a predetermined amount, for example 1% here, drives the illumination section 13 (S34), and the process moves to S30.

When the detected temperature is not higher than the predetermined temperature (S33: NO), the control section 21 stops driving the illumination section 13, then drives the image pickup section 12 and acquires a reference image (S36). As described above, the control section 21 heats the image pickup section 12, sets the temperature of the image pickup section 12 to a predetermined temperature, drives, when the temperature of the image pickup section 12 reaches the predetermined temperature, the image pickup section 12 and acquires a reference image.

Here, the acquisition of the reference image includes acquisition of a fixed pattern noise image to correct a sensitivity variation between pixels and acquisition of position information of defective pixels on the image pickup surface and the luminance level. Therefore, the reference image includes the fixed pattern noise image, the position information of defective pixels and the luminance level information.

The acquisition of the reference image may be acquisition of any one of the fixed pattern noise image, position information of defective pixels and the luminance level information, depending on the required image quality or the like.

Furthermore, if the result in S33 is NO, the reference image is immediately acquired in S36, and therefore after stopping driving the illumination section 13 (S35), the process moves to S36.

Furthermore, as described above, when a light-shielding seal member is pasted to the distal end portion 8, if the light-shielding performance of the seal member is high, driving of the illumination section 13 need not be stopped when the reference image is acquired.

The control section 21 stores and records the reference image acquired by image pickup by the image pickup section 12 in the memory 27 (S37).

The reference image may be acquired a plurality of times by the image pickup device and data of an average value or maximum value of the plurality of images acquired may be used as the reference image.

Moreover, the control section 21 may store the reference image not in the memory 27 but in the memory 14.

As described above, the control section 21 displays on the screen 5a of the display section 5 a predetermined message to announce a light-shielding instruction of the distal end portion 8 and drives the illumination section 13 after displaying the predetermined message, thereby heats the image pickup device, turns off the illumination section when the image pickup device is heated to the predetermined temperature, drives the image pickup device, acquires the reference image and stores the reference image in a predetermined memory.

After the reference image is stored, the control section 21 displays on the screen 5a of the display section 5 an “OK” button together with a message “Scope initialization is completed normally” (S38).

When the user selects the “OK” button on the screen (S39: YES), the control section 21 drives the illumination section 13 to turn it on, and the process moves to a normal operation state, that is, a state in which a live image is displayed (S40), and then the user can use the endoscope apparatus 1A for an inspection operation.

The reference image recorded in the memory 27 of the main body 4 is used by the video signal processing section 22 of the main body 4 to correct a sensitivity variation between pixels and pixel defects or the like, and clear video signals whose sensitivity variation between pixels or the like is corrected are outputted to the screen 5a of the display section 5.

Conventionally, the reference image is acquired in a thermostatic chamber in a factory or the like during manufacturing of the scope, and cannot be acquired accurately and reliably by the user.

According to the endoscope apparatus of the present embodiment, the user can acquire a reference image accurately and simply.

### 3. Modification Examples

Next, modification examples of the aforementioned second embodiment will be described.

### Modification Example 1

According to the aforementioned embodiment, the initialization processing is performed in the case where a different scope is detected when the power to the endoscope apparatus 1A is turned on first and a different scope is detected, and in the case where no different scope is detected and initialization is instructed.

Without being limited thereto, however, a menu screen may be displayed separately when the power to the endoscope apparatus 1A is turned on and the endoscope apparatus 1 is operating in a normal operation state, that is, while a live image is being displayed on the screen 5a of the display section 5, and the user may be instructed to select initialization from the menu screen so that the initialization processing from S25 onward is performed.

### Modification Example 2

According to the aforementioned embodiment, a new scope is attached after turning off the power to the endoscope apparatus 1A.

Without being limited thereto, however, in the case of an endoscope apparatus that allows the scope 7 to be removed or attached without turning off the power to the endoscope apparatus 1A, that is, in a hot line condition, the attachment of the scope after removal may be detected and the aforementioned initialization processing may be performed.

### Modification Example 3

According to the aforementioned embodiment, the illumination section 13 is driven and the image pickup section 12 is heated using the heat generated in the illumination section 13.

Without being limited thereto, however, as shown by a single-dot dashed line in FIG. 12, a heating section 16 for heating the image pickup section 12 may be provided separately aside from the illumination section 13 and the image pickup section 12 may be heated by driving the heating section 16.

The heating section 16 is configured by including a heating element provided in the vicinity of the image pickup device of the image pickup section 12 in the distal end portion 8 to heat the image pickup section 12. The heating section 16 is driven to generate heat based on a drive signal from the main body 4.

In that case, the main body 4 is provided with a heating drive section 28 that performs driving under the control of the control section 21 as shown by the single-dot dashed line in FIG. 12. The heating drive section 28 outputs a drive signal to the heating section 16 based on the control signal from the control section 21.

The heating section 16 and the heating drive section 28 are connected via a signal line and the control section 21 can control the heat of the heating section 16 by controlling the heating drive section 28.

### Modification Example 4

As a further modification example, the image pickup section 12 may be heated by driving the image pickup section 12 with a drive signal in a predetermined mode that can increase the temperature of the image pickup section 12 to a predetermined temperature.

The drive signal in this case is a drive signal based on such a drive scheme that allows the temperature of the image pickup section 12 to rise to a predetermined temperature and that increases power consumption in the image pickup section 12. That is, the image pickup section 12 can be heated by driving the image pickup section 12 with a drive signal in a high power consumption mode.

When the temperature of the image pickup section 12 reaches a predetermined temperature, the image pickup section 12 is driven not with the drive signal in the high power consumption mode but with a drive signal to acquire a normal reference image to acquire a reference image.

### Modification Example 5

According to the aforementioned embodiment, the illumination section 13 is driven and the image pickup section 12 is heated using heat generated by the illumination section 13. For example, in above-described modification example 3, the heating section 16 is provided separately and the image pickup section 12 is heated by driving the heating section 16. Furthermore, in above-described modification example 4, the image pickup section 12 is heated by driving the image pickup section 12 with a drive signal in a predetermined mode.

In the present modification example, the image pickup section 12 is heated by combining two or more of these three heating mechanisms or heating methods. The image pickup section 12 can be heated by using at least one of these heating methods.

Thus, the image pickup section 12 may be heated by combining two or more of these three heating mechanisms or heating methods.

### Modification Example 6

Furthermore, in the aforementioned embodiment, the temperature of the image pickup section 12 is detected by the temperature detection section 15.

Without being limited thereto, however, the temperature may also be detected by estimating the temperature of the image pickup section based on a video signal of an optical black part of the image pickup device. Since the video signal outputted from the image pickup device has a temperature characteristic in this way, the temperature of the image pickup section 12 can be estimated using a video image signal of the optical black part.

In such a case, the processing in S31 and S33 in FIG. 13 is a determination as to whether the signal level of the optical black part is lower or higher than a predetermined level SL.

### THIRD EMBODIMENT

Next, a third embodiment of the present invention will be described.

In the second embodiment, the user attaches the light-shielding adapter or the seal member to the distal end portion 8 of the scope 7 or inserts the distal end portion 8 into a dark box, but in the third embodiment, a shutter section is provided at the distal end portion 8 of the scope 7 and the image pickup section 12 is light-shielded using the shutter section.

### 1. Configuration

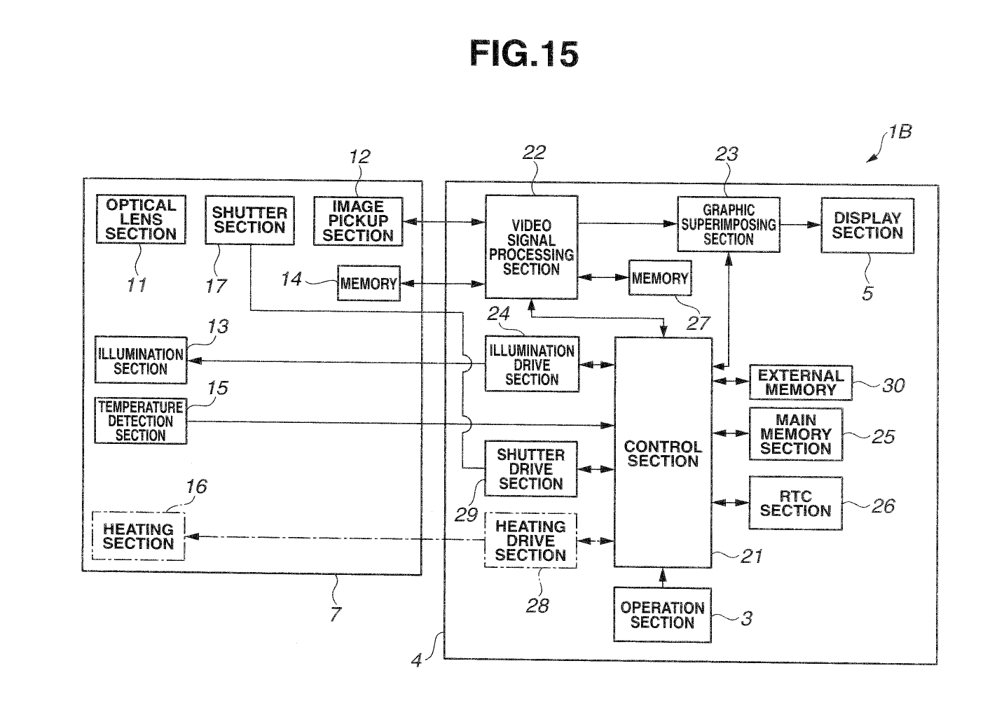

FIG. 15 is a block diagram schematically showing a configuration of an endoscope apparatus 1B according to the present embodiment. In FIG. 15, the same components as those in FIG. 12 will be assigned the same reference numerals and descriptions thereof will be omitted, and different components and operations will be described.

As shown in FIG. 15, the scope 7 has a shutter section 17. The shutter section 17 is placed between the optical lens section 11 and the image pickup section 12, and is configured and arranged so that when the shutter of the shutter section 17 is closed, light does not enter the image pickup section 12. When the shutter of the shutter section 17 is opened, reflected light from an object impinges on the image pickup section 12 via the optical lens section 11.

For example, the shutter section 17 has a mechanical mechanism whereby a light-shield sheet driven by a micro motor or the like can cover the front surface of the image pickup section 12.

Furthermore, the main body 4 has a shutter drive section 29 and the shutter section 17 is opened/closed based on a drive signal from the shutter drive section 29. The shutter drive section 29 outputs a drive signal for opening/closing the shutter section 17 to the shutter section 17 based on a control signal from the control section 21.

### 2. Operation

Next, an initialization operation in the endoscope apparatus 1B will be described.

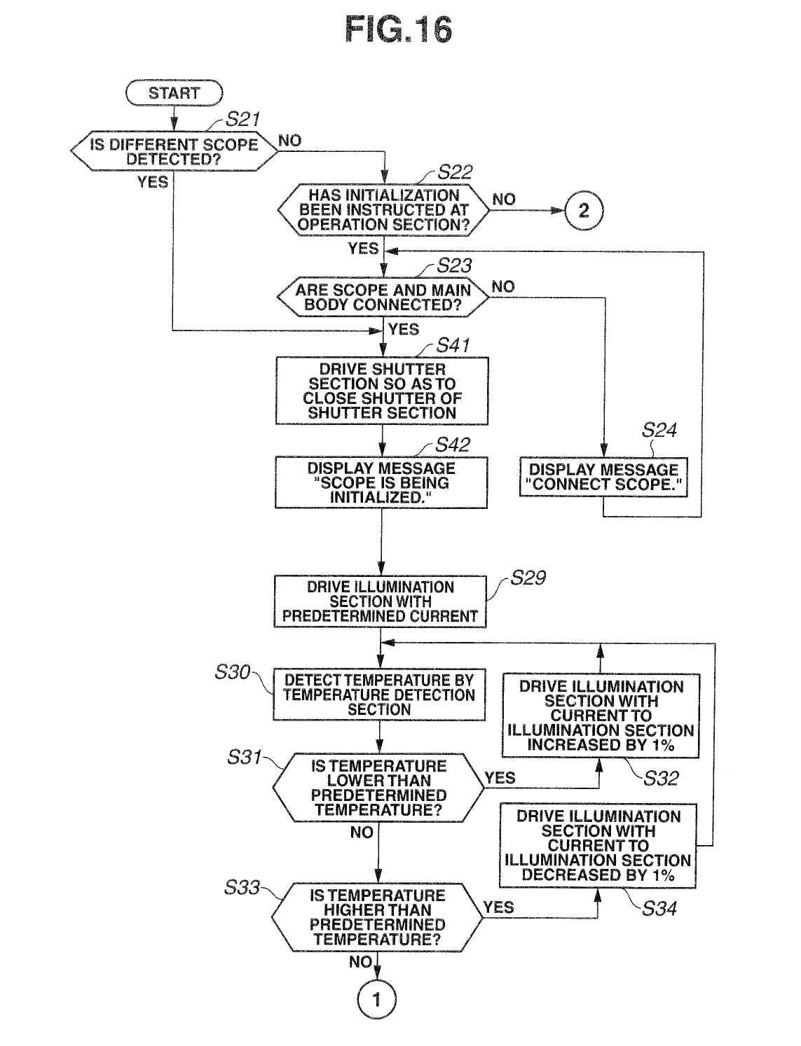

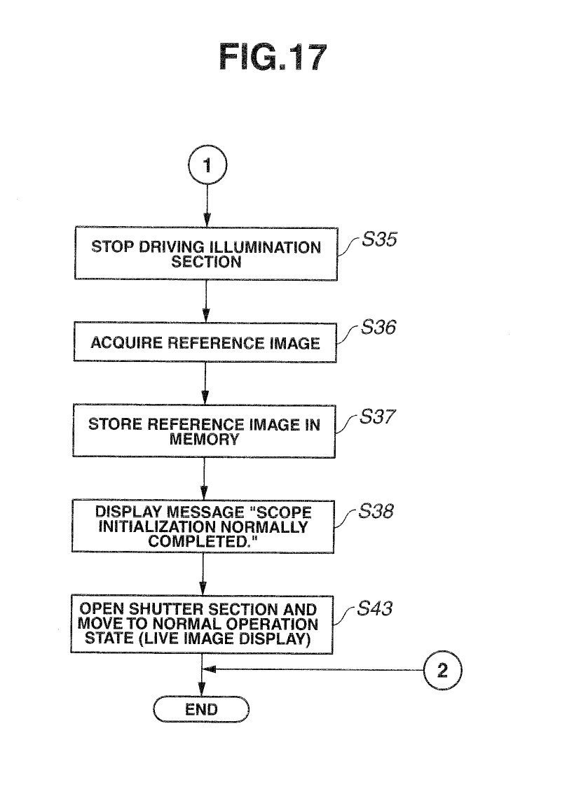

FIG. 16 and FIG. 17 are flowcharts illustrating an example of part of a processing flow of an initialization operation of the endoscope apparatus 1B. In FIG. 16 and FIG. 17, the same processes as those in the second embodiment will be assigned the same reference numerals and descriptions thereof will be omitted and only parts of different processes will be described.

As shown in FIG. 16, when different scopes are detected and when initialization is instructed (after processes in S21 and S23), the control section 21 generates and transmits a drive signal to close the shutter of the shutter section 17 and thereby drives the shutter section 17 so as to close the shutter of the shutter section 17 (S41). This allows the image pickup section 12 to be automatically light-shielded without the user performing a light-shielding operation using a light-shielding member or the like as in the case of the second embodiment.

The control section 21 then displays on the screen 5a of the display section 5 a message (not shown) “Scope initialization is in progress” (S42) and the process moves to S29. The subsequent processes are similar to the process described in the second embodiment.

After the process in S38, the control section 21 generates and transmits a drive signal to open the shutter of the shutter section 17, thereby drives the shutter section 17 so as to open the shutter of the shutter section 17 and the process moves to a normal operation state (S43).

Therefore, the endoscope apparatus of the present embodiment can also implement an endoscope apparatus that allows the user to accurately and simply acquire a reference image.

The modification examples described in the second embodiment are also applicable to the present third embodiment.

Conventionally, the reference image is acquired in a thermostatic chamber in a factory or the like during manufacturing of the scope, and cannot be acquired accurately and simply by the user.

According to the embodiments and modification examples, the user can accurately and simply acquire a reference image.

The present invention is not limited to the aforementioned embodiments, but various modifications and alterations or the like can be made without departing from the spirit and scope of the present invention.

What is claimed is:

1. An endoscope apparatus comprising:
a main body to which a scope having an elongated insertion portion and an image pickup device at a distal end portion of the insertion portion is detachably attachable;
a video signal processing section, provided in the main body, which drives the image pickup device and processes a video signal acquired by image pickup through the image pickup device;
a display section; and
a control section, provided in the main body, which displays on the display section a first message for instructing a user to perform light shielding of the distal end portion according to an instruction for starting initialization processing to acquire a reference image of the image pickup device, acquires the reference image with the distal end portion light-shielded and stores the acquired reference image in a predetermined memory.

2. The endoscope apparatus according to claim 1, wherein the control section displays the first message on the display section, and then displays a second message announcing to the user that the initialization processing is in progress before acquiring the reference image and acquires the reference image with the distal end portion light-shielded.

3. The endoscope apparatus according to claim 1, wherein the control section acquires the reference image with the distal end portion light-shielded, and then displays on the display section a sixth message announcing to the user that the initialization processing is completed.

4. The endoscope apparatus according to claim 1, wherein the control section displays the first message, then determines a light-shielding state of the distal end portion and displays, upon determining from the determination of the light-shielding state that the distal end portion is not correctly light-shielded, a third message for instructing that the distal end portion be correctly light-shielded on the display section.

5. The endoscope apparatus according to claim 1, wherein the control section displays on the display section a fourth message that announces a time period required to acquire the reference image to the user.

6. The endoscope apparatus according to claim 1, wherein upon detecting that the scope is not connected to the main body, the control section displays on the display section a fifth message for instructing that the scope be connected to the main body before acquiring the reference image.

7. The endoscope apparatus according to claim 1, wherein the control section displays on the display section an initialization completion screen for announcing to the user that the initialization processing is completed after the reference image is stored in the predetermined memory.

8. The endoscope apparatus according to claim 1, wherein the control section displays the initialization instruction screen on the display section upon detecting that the scope connected when power to the main body of the endoscope apparatus is switched on is different from the scope connected when the main body is switched off last time.

9. The endoscope apparatus according to claim 1, wherein the initialization instruction screen is a menu screen.

10. The endoscope apparatus according to claim 1, wherein the control section displays the initialization instruction screen on the display section when the scope is replaced while power to the main body of the endoscope apparatus is on.

11. The endoscope apparatus according to claim 5, wherein the time period required to acquire the reference image includes a time period until the image pickup device is heated by a heater provided at the distal end portion of the scope to a predetermined temperature.

12. The endoscope apparatus according to claim 1, wherein the predetermined memory is provided in the main body.

13. A method of setting a reference image of an endoscope apparatus, comprising:
displaying on a display section a first message for instructing a user to perform light-shielding of a distal end portion of a scope which is detachably attachable to a main body of the endoscope apparatus according to an instruction for starting initialization processing to acquire a reference image of an image pickup device provided at the distal end portion;
acquiring the reference image with the distal end portion light-shielded; and
storing the acquired reference image in a predetermined memory.

14. The method of setting a reference image of an endoscope apparatus according to claim 13, wherein after the first message is displayed on the display section,
a second message for announcing to the user that the initialization processing is in progress is displayed on the display section before acquiring the reference image, and
the reference image is acquired with the distal end portion light-shielded.

15. The method of setting a reference image of an endoscope apparatus according to claim 13, wherein the reference image is acquired with the distal end portion light-shielded, and
when the initialization processing is completed, a sixth message annunciating to the user that the initialization processing is completed is displayed on the display section.

16. The method of setting a reference image of an endoscope apparatus according to claim 13, wherein a light-shielding state of the distal end portion is determined after the first message is displayed, and
when the determination result of the light-shielding state shows that the distal end portion is not correctly light-shielded, a third message for instructing that the distal end portion be correctly light-shielded is displayed on the display section.

17. The method of setting a reference image of an endoscope apparatus according to claim 13, wherein a fourth message for announcing a time period required to acquire the reference image to the user is displayed on the display section.

18. The method of setting a reference image of an endoscope apparatus according to claim 13, wherein before the reference image is acquired if the scope is detected not to be connected to the main body, a fifth message for instructing that the scope be connected to the main body is displayed on the display section.

19. The method of setting a reference image of an endoscope apparatus according to claim 13, wherein after the reference image is stored in the predetermined memory, an initialization completion screen for announcing completion of the initialization processing to the user is displayed on the display section.

20. The method of setting a reference image of an endoscope apparatus according to claim 13, wherein the predetermined memory is provided in the main body.

21. An endoscope apparatus comprising:
a main body, to which a scope having an image pickup device at a distal end portion of an insertion portion is detachably attachable;
a video signal processing section, provided in the main body, which drives the image pickup device and processes a video signal acquired by image pickup through the image pickup device; and
a control section that displays on a display section a predetermined message to announce an instruction of light shielding of the distal end portion, heats the image pickup device after the predetermined message is displayed, drives the image pickup device with the temperature of the image pickup device kept at a predetermined temperature, acquires a reference image and stores the reference image in a predetermined memory.

22. The endoscope apparatus according to claim 21, wherein the control section heats the image pickup device by driving an illumination section provided at the distal end portion of the insertion portion.

23. The endoscope apparatus according to claim 21, wherein the control section heats the image pickup device by driving a heating section provided at the distal end portion of the insertion portion to generate heat.

24. The endoscope apparatus according to claim 21, wherein the control section heats the image pickup device by driving the image pickup device in a predetermined mode to generate heat.

25. A method of acquiring a reference image of an endoscope apparatus comprising:
displaying on a display section a message for instructing light shielding of an image pickup section provided at a distal end portion of a scope so as to prevent light from entering the image pickup section;
heating the image pickup section so that a temperature of the image pickup section reaches a predetermined temperature;
driving the image pickup section when the temperature of the image pickup section reaches the predetermined temperature to acquire a reference image; and
storing the acquired reference image in a predetermined memory.

26. The method of acquiring a reference image of an endoscope apparatus according to claim 25, wherein the temperature of the image pickup section is set to the predetermined temperature by performing at least one of:
driving an illumination section provided at the distal end portion;
driving a heating section provided at the distal end portion; and
driving the image pickup section in a predetermined mode.
